(19) United States
(12) Patent Application Publication
(10) Pub. No.: US 2024/0203543 A1
(43) Pub. Date: Jun. 20, 2024

(54) DEEP LEARNING-BASED METHOD AND DEVICE FOR PREDICTING ANALYSIS RESULTS

(71) Applicants: Kwangwoon University Industry-Academic Collaboration Foundation, Seoul (KR); CATHOLIC KWANDONG UNIVERSITY INDUSTRY FOUNDATION, Gangneung-si (KR)

(72) Inventors: Jeong Hoon LEE, Seongnam-si (KR); Ki Baek LEE, Namyangju-si (KR); Yong Kyoung YOO, Seoul (KR); Seung Min LEE, Seoul (KR); Hak Jun LEE, Seoul (KR); Ji Won MOON, Seoul (KR)

(21) Appl. No.: 18/595,267

(22) Filed: Mar. 4, 2024

Related U.S. Application Data
(63) Continuation-in-part of application No. PCT/KR2022/002020, filed on Feb. 10, 2022.

(30) Foreign Application Priority Data
Sep. 2, 2021 (KR) 10-2021-0117172

(51) Int. Cl. G16H 10/40

(52) U.S. Cl. CPC G16H 10/40

(57) ABSTRACT

According to a preferred embodiment of the present invention, a deep learning-based method and device for predicting analysis results predict analysis results of immune response assay-based kits, such as that of lateral flow assay (LEA), antigen-antibody-based diagnostic kits and the like on the basis of deep learning, and thus can reduce the time for confirming results.

### CROSS-REFERENCE TO RELATED APPLICATIONS

This application is a Continuation-in-Part of PCT International Application No. PCT/KR2022/002020, filed on Feb. 10, 2022, which claims priority to Korean Patent Application No. 10-2021-0117172 filed on Sep. 2, 2021, the entire contents of which are hereby incorporated by references in its entirety.

### TECHNICAL FIELD

The present invention relates to a deep learning-based method and a device for predicting analysis results, and more particularly, to a method and a device for predicting analysis results based on deep learning.

### BACKGROUND ART

It takes a long time (10 to 30 minutes) for a diagnostic test through a lateral flow assay (LFA) reaction which collects samples from a specimen and uses the collected samples. The lateral flow assay shows different aspects depending on a sample concentration and a reaction time and the diagnostic test using the lateral flow assay (LFA) reaction can only be determined when a sufficient reaction occurs after approximately 15 minutes. However, in the case of certain diseases, such as myocardial infarction, results are frequently requested within 10 minutes and recently, the need for a quick diagnosis within five minutes has increased significantly from the perspective of hospitals and patients.

### DISCLOSURE

### Technical Problem

An object to be achieved by the present invention is to provide a deep learning-based analysis result predicting method and device which predict analysis results of immune response assay-based kit such as a lateral flow assay (LEA) and antigen-antibody-based diagnostic kits on the basis of deep learning.

Other and further objects of the present invention which are not specifically described can be further considered within the scope easily deduced from the following detailed description and the effect.

### Technical Solution

In order to achieve the above-described object, according to a preferred embodiment of the present invention, a deep learning-based analysis result predicting method includes a step of obtaining a reaction image for a predetermined initial period for an interaction of a sample obtained from a specimen and an optical-based kit; and a step of predicting a concentration for a predetermined result time on the basis of the reaction image for the predetermined initial period, using a pre-trained and established analysis result prediction model.

Here, the step of obtaining a reaction image is configured by obtaining a plurality of reaction images in a predetermined time unit for the predetermined initial period.

Here, the analysis result prediction model includes: an image generator includes a convolution neural network (CNN), long short-term memory (LSTM), and a generative adversarial network (GAN), and generates a prediction image corresponding to a predetermined result time on the basis of an input reaction image, and outputs the generated prediction image; and a regression model which includes the convolution neural network (CNN) and outputs a predicted concentration for a predetermined result time on the basis of the prediction image generated by the image generator, and the regression model is trained using the learning data to minimize the difference of the predicted concentration for the predetermined result time obtained on the basis of the reaction image of the learning data and the actual concentration for the predetermined result time of the learning data.

Here, the image generator includes: an encoder which obtains a feature vector from the input reaction image using the convolution neural network (CNN), obtains a latent vector on the basis of the obtained feature vector using the long short term memory (LSTM), and outputs the obtained latent vector; and a decoder which generates the prediction image on the basis of the latent vector obtained from the encoder using the generative adversarial network (GAN), and outputs the generated prediction image.

Here, the decoder includes a generator which generates the prediction image on the basis of the latent vector and outputs the generated prediction image; and a discriminator which compares the prediction image generated by the generator and an actual image corresponding to a predetermined result time of the learning data and outputs a comparison result, and it is trained to discriminate that the prediction image obtained on the basis of the latent vector is the actual image using the learning data.

Here, the step of obtaining a reaction image is configured by obtaining the reaction image of an area corresponding to the test-line when the optical-based kit includes a test-line and a control-line.

Here, the step of obtaining a reaction image is configured by obtaining the reaction image of the area corresponding to one or more predetermined test-lines, among a plurality of test-lines when the optical-based kit includes a plurality of test-lines.

Here, the step of obtaining a reaction image is configured by obtaining the reaction image including all areas corresponding to one or more predetermined test-lines, among the plurality of test-lines or obtaining the reaction image for every test-line to distinguish areas corresponding to one or more predetermined test-lines, among the plurality of test-lines for every test-line.

In order to achieve the above-described technical object, according to a preferred embodiment of the present invention, a computer program is stored in a computer readable storage medium to allow a computer to execute any one of the deep learning-based analysis result predicting methods.

In order to achieve the above-described object, according to a preferred embodiment of the present invention, a deep learning-based analysis result predicting device is a deep learning-based analysis result predicting device which predicts an analysis result based on deep learning and includes a memory which stores one or more programs to predict an analysis result; and one or more processors which perform an operation for predicting the analysis result according to one or more programs stored in the memory, and the processor predicts a concentration for a predetermined result time on the basis of a reaction image of a predetermined initial period for an interaction of a sample obtained from a specimen and an optical-based kit, using a pre-trained and established analysis result prediction model.

Here, the processor obtains a plurality of reaction images in a predetermined time unit for the predetermined initial period.

Here, the analysis result prediction model includes: an image generator includes a convolution neural network (CNN), long short-term memory (LSTM), and a generative adversarial network (GAN), and generates a prediction image corresponding to a predetermined result time on the basis of an input reaction image, and outputs the generated prediction image; and a regression model which includes the convolution neural network (CNN) and outputs a predicted concentration for a predetermined result time on the basis of the prediction image generated by the image generator, and the regression model is trained using the learning data to minimize the difference of the predicted concentration for the predetermined result time obtained on the basis of the reaction image of the learning data and the actual concentration for the predetermined result time of the learning data.

Here, the image generator includes: an encoder which obtains a feature vector from the input reaction image using the convolution neural network (CNN), obtains a latent vector on the basis of the obtained feature vector using the long short term memory (LSTM), and outputs the obtained latent vector; and a decoder which generates the prediction image on the basis of the latent vector obtained from the encoder using the generative adversarial network (GAN), and outputs the generated prediction image.

### Advantageous Effects

According to the deep learning-based analysis result predicting method and device according to a preferred embodiment of the present invention, analysis results of immune response assay-based kit such as a lateral flow assay (LEA) and antigen-antibody-based diagnostic kits are predicted on the basis of deep learning to reduce the time for confirming results.

The effects of the present disclosure are not limited to the technical effects mentioned above, and other effects which are not mentioned can be clearly understood by those skilled in the art from the following description

### BRIEF DESCRIPTION OF DRAWINGS

FIG. 1 is a block diagram for explaining a deep learning-based analysis result predicting device according to a preferred embodiment of the present invention.

FIG. 2 is a view for explaining a process of predicting analysis results according to a preferred embodiment of the present invention.

FIG. 3 is a view for explaining a process of predicting changes of color intensity over time based on a reaction image according to a preferred embodiment of the present invention.

FIG. 4 is a view for explaining a process of predicting a concentration for a result time on the basis of a reaction image for an initial period according to a preferred embodiment of the present invention.

FIG. 5 is a flowchart for explaining a deep learning-based analysis result predicting method according to a preferred embodiment of the present invention.

FIG. 6 is a view for explaining an example of a structure of an analysis result prediction model according to a preferred embodiment of the present invention.

FIG. 7 is a view for explaining an implementation example of an analysis result prediction model illustrated in FIG. 6.

FIG. 8 is a view for explaining another example of a structure of an analysis result prediction model according to a preferred embodiment of the present invention.

FIG. 9 is a view for explaining learning data used for a learning process of an analysis result prediction model according to a preferred embodiment of the present invention.

FIG. 10 is a view for explaining a configuration of learning data illustrated in FIG. 9.

FIG. 11 is a view for explaining an example of a reaction image illustrated in FIG. 10.

FIG. 12 is a view for explaining an example of a pre-processing process of a reaction model according to a preferred embodiment of the present invention.

FIG. 13 illustrates three representative commercialized diagnostic tools.

FIG. 14 illustrates an example of a deep learning architecture,

FIG. 15 illustrates an assessment of infectious diseases, specifically COVID-19 antigen and Influenza A/B, using a 2-minute assay facilitated by the TIMESAVER model.

FIG. 16 illustrates an assessment of non-infectious biomarkers for emergency room (ER) via the TIMESAVER model.

FIG. 17 illustrates the clinical evaluation of COVID-19 through blind tests.

### MODE FOR CARRYING OUT THE DISCLOSURE

Hereinafter, embodiments of the present invention will be described in detail with reference to the accompanying drawings. Advantages and characteristics of the present invention and a method of achieving the advantages and characteristics will be clear by referring to preferable embodiments described below in detail together with the accompanying drawings. However, the present invention is not limited to preferable embodiments disclosed herein, but will be implemented in various different forms. The preferable embodiments are provided by way of example only so that a person of ordinary skilled in the art can fully understand the disclosures of the present invention and the scope of the present invention. Therefore, the present invention will be defined only by the scope of the appended claims. Like reference numerals generally denote like elements throughout the specification.

Unless otherwise defined, all terms (including technical and scientific terms) used in the present specification may be used as the meaning which may be commonly understood by the person with ordinary skill in the art, to which the present disclosure belongs. It will be further understood that terms defined in commonly used dictionaries should not be interpreted in an idealized or excessive sense unless expressly and specifically defined.

In the specification, the terms “first” and “second” are used to distinguish one component from the other component so that the scope should not be limited by these terms. For example, a first component may also be referred to as a second component and likewise, the second component may also be referred to as the first component.

In the present specification, in each step, numerical symbols (for example, a, b, and c) are used for the convenience of description, but do not explain the order of the steps so that unless the context apparently indicates a specific order, the order may be different from the order described in the specification. In the present specification, in each step, numerical symbols (for example, a, b, and c) are used for the convenience of description, but do not explain the order of the steps so that unless the context apparently indicates a specific order, the order may be different from the order described in the specification.

In this specification, the terms “have”, “May have”, “include”, or “May include” represent the presence of the characteristic (for example, a numerical value, a function, an operation, or a component such as a part”), but do not exclude the presence of additional characteristic.

Hereinafter, a preferred embodiment of a deep learning based analysis result predicting method and device according to the present invention will be described in detail with reference to the accompanying drawings.

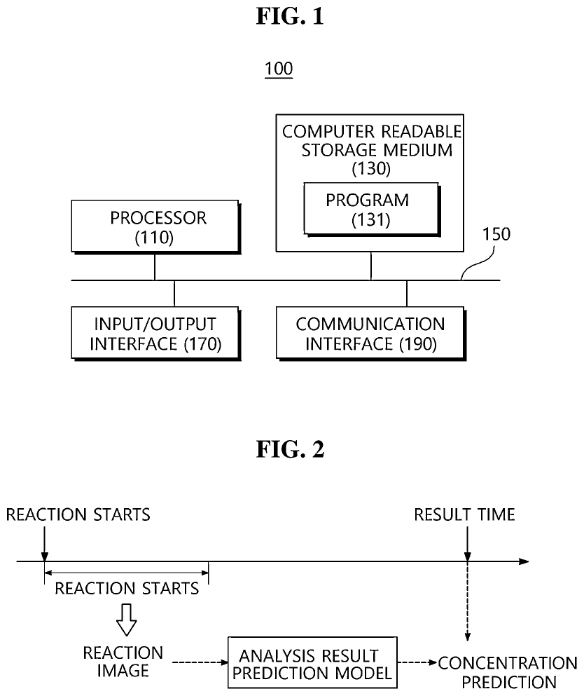

First, a deep learning based analysis result predicting device according to the present invention will be described with reference to FIGS. 1 to 4.

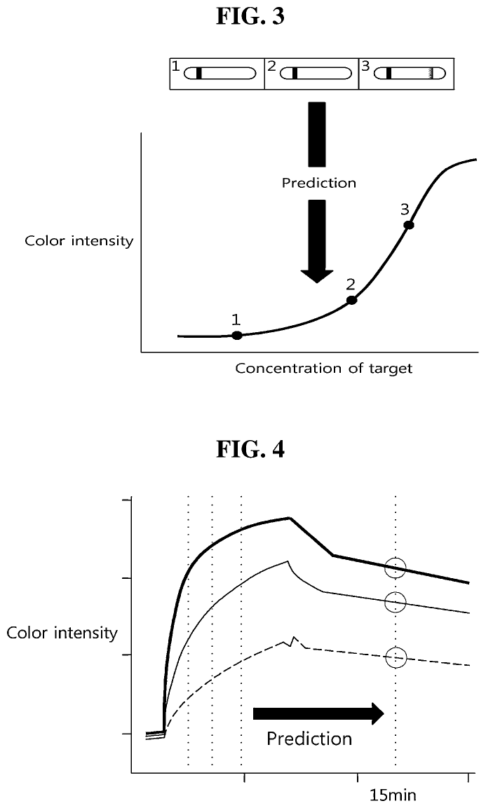

FIG. 1 is a block diagram for explaining a deep learning-based analysis result predicting device according to a preferred embodiment of the present invention, FIG. 2 is a view for explaining a process of predicting analysis results according to a preferred embodiment of the present invention, FIG. 3 is a view for explaining a process of predicting changes of color intensity over time based on a reaction image according to a preferred embodiment of the present invention, and FIG. 4 is a view for explaining a process of predicting a concentration for a result time on the basis of a reaction image for an initial period according to a preferred embodiment of the present invention.

Referring to FIG. 1, a deep learning-based analysis result predicting device according to a preferred embodiment of the present invention (hereinafter, referred to as an “analysis result predicting device) 100 predicts analysis results of immune response assay-based kits such as lateral flow assay (LFA) and antigen-antibody-based diagnostic kits on the basis of deep learning.

In the meantime, an operation of predicting analysis results on the basis of deep learning according to the present invention may be applied not only to lateral flow assay which derives a result on the basis of an color intensity, but also to another analysis which derives a result on the basis of fluorescence intensity. However, for the convenience of description of the present invention, the following description will be made under the assumption that the present invention predicts lateral flow assay results.

To this end, the analysis result predicting device 100 may include one or more processors 110, a computer readable storage medium 130, and a communication bus 150.

The processor 110 controls the analysis result predicting device 100 to operate. For example, the processor 110 may execute one or more programs 131 stored in the computer readable storage medium 130. One or more programs 131 include one or more computer executable instructions and when the computer executable instruction is executed by the processor 110, the computer executable instruction may be configured to allow the analysis result predicting device 100 to perform an operation for predicting a result of analysis (for example, lateral flow assay).

The computer readable storage medium 130 is configured to store a computer executable instruction or program code, program data and/or other appropriate format of information to predict a result of analysis (for example, lateral flow assay). The program 131 stored in the computer readable storage medium 130 includes a set of instructions executable by the processor 110. In one preferable embodiment, the computer readable storage medium 130 may be a memory (a volatile memory such as a random access memory, a non-volatile memory, or an appropriate combination thereof), one or more magnetic disk storage devices, optical disk storage devices, flash memory devices, and another format of storage media which are accessed by the analysis result predicting device 100 and store desired information, or an appropriate combination thereof.

The communication bus 150 interconnects various other components of the analysis result predicting device 100 including the processor 110 and the computer readable storage medium 130 to each other.

The analysis result predicting device 100 may include one or more input/output interfaces 170 and one or more communication interfaces 190 which provide an interface for one or more input/output devices. The input/output interface 170 and the communication interface 190 are connected to the communication bus 150. The input/output device (not illustrated) may be connected to another components of the analysis result predicting device 100 by means of the input/output interface 170.

Referring to FIG. 2, the processor 110 of the analysis result predicting device 100 predicts a concentration for a predetermined result time based on a reaction image of a predetermined initial period for interaction of samples obtained from specimen and optical-based kits (for example, immune response assay-based kits such as lateral flow assay (LFA), or an antigen-antibody-based diagnostic kits, such as LSPR, SPR, and fluorescence based assay) using an analysis result prediction model which is pre-trained and established.

Here, the predetermined initial period and the predetermined result time may vary depending on a kind or a type of the optical based kit and specifically, the predetermined result time refers to a time that a final result for the sample is confirmed. For example, the predetermined result time may be set to “15 minutes” and the predetermined initial period may be set to “0 to 5 minutes”.

At this time, the processor 110 may obtain a plurality of reaction images in the predetermined time unit for the predetermined initial period. For example, when the predetermined initial period is “0 to 5 minutes” and the predetermined time unit is “10 seconds”, the processor 110 may obtain 30 (=6×5) reaction images.

The analysis result prediction model includes a convolution neural network (CNN), a long short-term memory (LSTM), and a generative adversarial network (GAN), and details thereof will be described below.

That is, as illustrated in FIG. 3, a change in color intensity over time may be predicted through a reaction image for interaction of the optical based kit including one test-line and one control-line and a sample at a specific timing. Accordingly, as illustrated in FIG. 4, a concentration for a predetermined result time (for example, 15 minutes) may be predicted through the reaction image for the predetermined initial period.

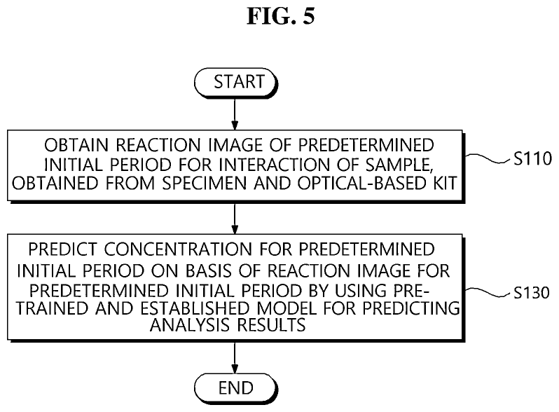

Now, a deep learning based analysis result predicting method according to the present invention will be described with reference to FIG. 5.

FIG. 5 is a flowchart for explaining a deep learning-based analysis result predicting method according to a preferred embodiment of the present invention.

Referring to FIG. 5, the processor 110 of the analysis result predicting device 100 obtain a reaction image of a predetermined initial period for an interaction of a sample obtained from a specimen and an optical based kit (S110).

At this time, the processor 110 may obtain a plurality of reaction images in the predetermined time unit for the predetermined initial period.

The processor 110 performs a pre-processing process of the reaction image before inputting the reaction image to the analysis result prediction model.

That is, when the optical-based kit includes a test-line and a control-line, the processor 110 obtains a reaction image of an area corresponding to the test-line. Here, the size of the area may be set in advance to be “200×412 size”.

In the meantime, when the optical-based kit includes a plurality of test-lines, the processor 110 may obtain a reaction image of an area corresponding to one or more predetermined test-lines, among the plurality of test-lines.

At this time, the processor 100 may obtain a reaction image including all the areas corresponding to one or more predetermined test-lines, among the plurality of test-lines or obtain a reaction image for every test-line to distinguish the areas corresponding to one or more predetermined test-lines, among the plurality of test-lines, for every test-line.

Thereafter, the processor 110 predicts a concentration for the predetermined result time on the basis of the reaction image for the predetermined initial period, using the pre-trained and established analysis result prediction model (S130).

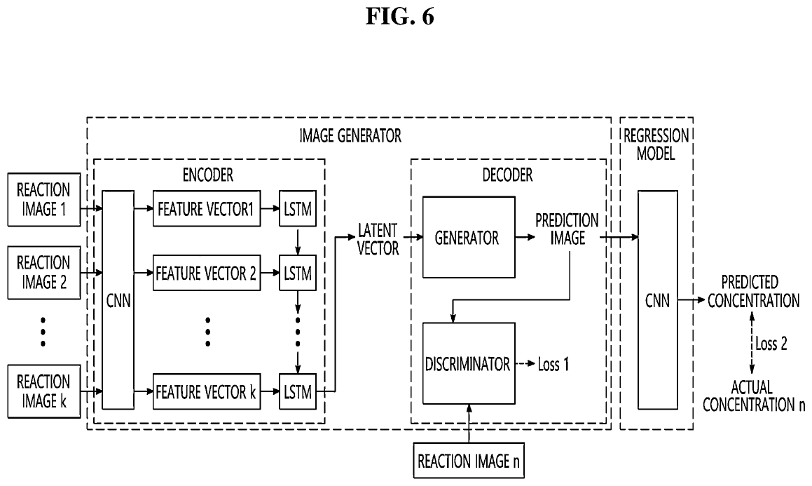

Now, a structure of the analysis result prediction model according to a preferred embodiment of the present invention will be described with reference to FIGS. 6 to 8.

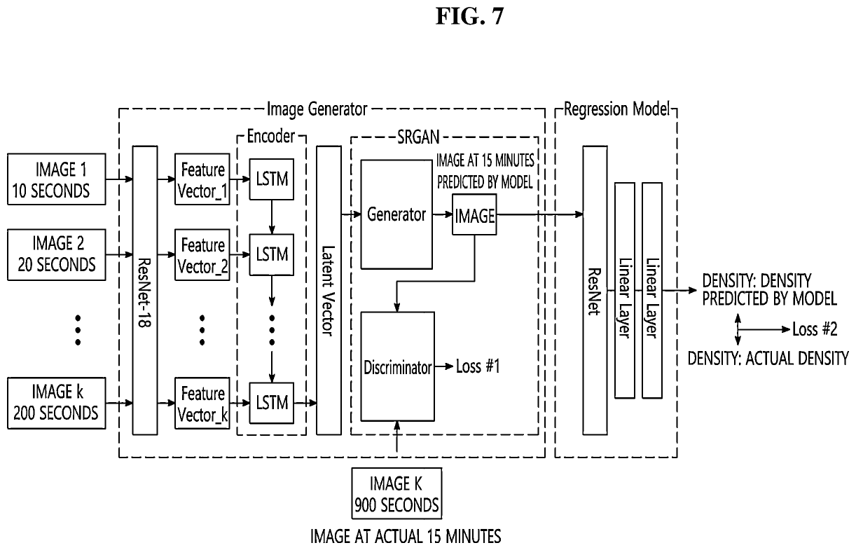

FIG. 6 is a view for explaining an example of a structure of an analysis result prediction model according to a preferred embodiment of the present invention and FIG. 7 is a view for explaining an implementation example of an analysis result prediction model illustrated in FIG. 6.

Referring to FIG. 6, an example of the analysis result prediction model according to the present invention includes an image generator and a regression model.

The image generator includes a convolution neural network (CNN), a long short-term memory (LSTM), and a generative adversarial network (GAN), generates a prediction image corresponding to a predetermined result time on the basis of a plurality of input reaction images, and outputs the generated prediction image.

To this end, the image generator includes an encoder and a decoder.

The encoder obtains a feature vector from each of the plurality of input reaction images using the convolution neural network (CNN), obtains a latent vector on the basis of the plurality of obtained feature vectors using the long short term memory (LSTM), and outputs the obtained latent vector. That is, the encoder calculates a relationship of a concentration, a variance of color intensity, and a time from the plurality of feature vectors to generate the latent vector.

The decoder generates a prediction image using the generative adversarial network

(GAN) on the basis of the latent vector obtained from the encoder, and outputs the generated prediction image.

That is, the decoder includes a generator which generates the prediction image on the basis of the latent vector and outputs the generated prediction image and a discriminator which compares a prediction image generated by the generator and an actual image corresponding to the predetermined result time of the learning data and outputs a comparison result.

At this time, the decoder is trained using the learning data so as to discriminate that the prediction image obtained based on the latent vector is an actual image.

A regression model includes a convolution neural network (CNN) and outputs a predicted concentration for a predetermined result time on the basis of the prediction image generated by the image generator. That is, the regression model obtains a feature vector of the prediction image and obtains a predicted concentration by causing the obtained feature vector to pass through two linear layers.

At this time, the regression model is trained using the learning data to minimize the difference of the predicted concentration for the predetermined result time obtained on the basis of the reaction image of the learning data and the actual concentration for the predetermined result time of the learning data.

In the meantime, the discriminator is a module required for a learning process of the analysis result prediction model and is removed from the analysis result prediction model after completing the learning.

For example, as illustrated in FIG. 7, when the plurality of reaction images (images 1 to k of FIG. 7) obtained at every 10 seconds for the initial period (10 to 200 seconds) is input to the analysis result prediction model, the analysis result prediction model obtains feature vectors (Feature Vector_1 to Feature Vector_k) using ResNet-18 which is a convolution neural network) from the plurality of reaction images (images 1 to k of FIG. 7). Thereafter, the analysis result prediction model inputs the plurality of obtained feature vectors (Feature Vector_1 to Feature Vector_k of FIG. 7) to a corresponding long short term memory (LSTM).

Thereafter, the analysis result prediction model inputs the latent vector output from the long short term memory (LSTM) to a generator of “SRGAN” which is a generative adversarial neural network (GAN). By doing this, the analysis result prediction model generates a prediction image corresponding to the result time (15 minutes) on the basis of the latent vector. Thereafter, the analysis result prediction model inputs the generated prediction image to discriminators of ResNet and SRGAN which are the convolution neural networks (CNN). Here, the discriminator compares the prediction image and the actual image corresponding to the result time (15 minutes) and provides the comparison result to the generator.

By doing this, the analysis result prediction model outputs a predicted concentration for the result time (15 minutes).

Here, the analysis result prediction model may be trained using the learning data to minimize two losses. A first loss (Loss #1 in FIG. 7) is to discriminate the prediction image obtained on the basis of the latent vector as an actual image. A second loss (Loss #2 in FIG. 7) is to minimize the difference between the predicted concentration for the result time (15 minutes) and the actual concentration for the result time (15 minutes).

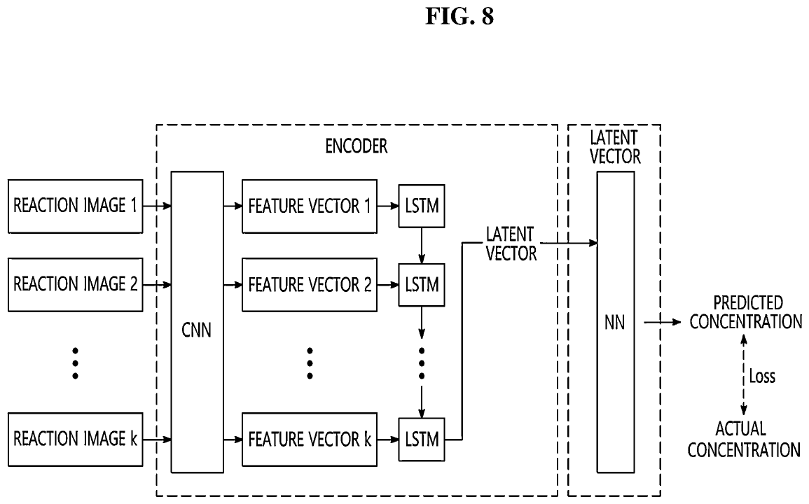

FIG. 8 is a view for explaining another example of a structure of an analysis result prediction model according to a preferred embodiment of the present invention.

Referring to FIG. 8, another example of the analysis result prediction model according to the present invention is substantially the same as an example (see FIG. 6) of the analysis result prediction model which has been described above and a process of generating an image is omitted from the example (see FIG. 6) of the analysis result prediction model.

That is, another example of the analysis result prediction model removes a decoder from the example (see FIG. 6) of the analysis result prediction model to include an encoder and a regression model.

The encoder includes a convolution neural network (CNN) and a long short term memory (LSTM) and generates a latent vector on the basis of the plurality of input reaction images and outputs the generated latent vector. In other words, the encoder obtains a feature vector from each of the plurality of input reaction images using the convolution neural network (CNN), obtains a latent vector on the basis of the plurality of obtained feature vectors using the long short term memory (LSTM), and outputs the obtained latent vector.

The regression model includes a neural network (NN) and outputs a predicted concentration for a predetermined result time on the basis of the latent vector obtained through the encoder. At this time, the regression model is trained using the learning data to minimize the difference of the predicted concentration for the predetermined result time obtained on the basis of the reaction image of the learning data and the actual concentration for the predetermined result time of the learning data.

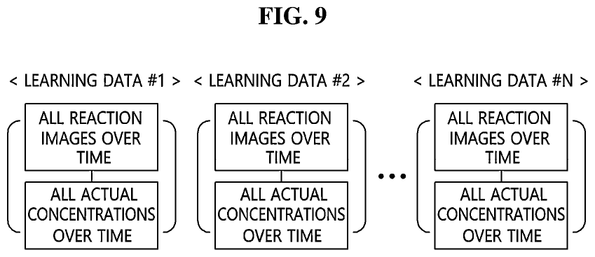

Now, learning data used for a training process of the analysis result prediction model according to a preferred embodiment of the present invention will be described with reference to FIGS. 9 to 12.

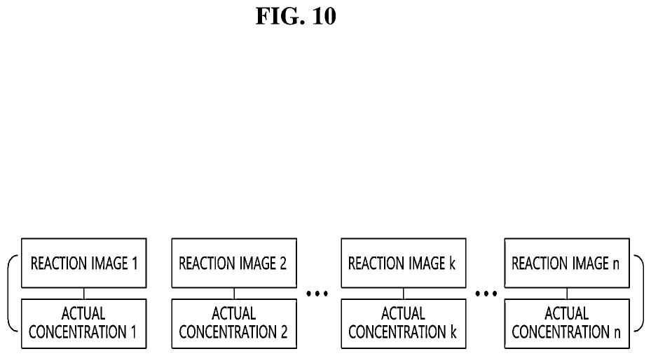

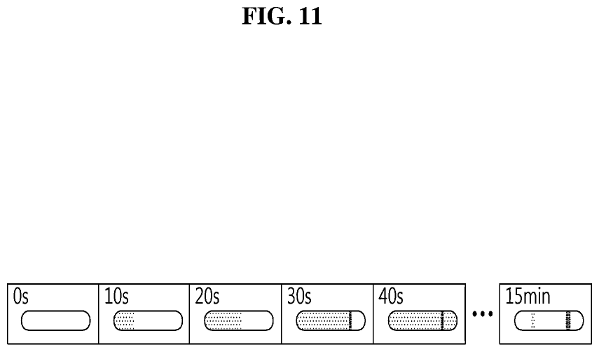

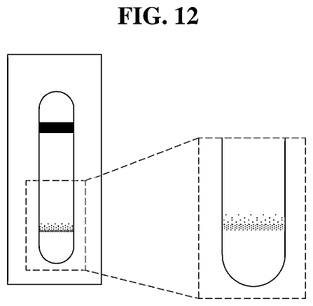

FIG. 9 is a view for explaining learning data used for a learning process of an analysis result prediction model according to a preferred embodiment of the present invention, FIG. 10 is a view for explaining a configuration of learning data illustrated in FIG. 9, FIG. 11 is a view for explaining an example of a reaction image illustrated in FIG. 10, and FIG. 12 is a view for explaining an example of a pre-processing process of a reaction model according to a preferred embodiment of the present invention.

The learning data used for the learning process of the analysis result prediction model according to the present invention may be configured by a plurality of data as illustrated in FIG. 9.

That is, as illustrated in FIG. 10, each learning data includes all reaction images (reaction image 1 to reaction image n in FIG. 10) over time and all actual concentrations (actual concentration 1 to actual concentration (actual concentration 1 to actual concentration n of FIG. 10) over time. Here, “Reaction Image 1 to Reaction image k” illustrated in FIG. 10 indicate reaction images of the initial period. For example, as illustrated in FIG. 11, the reaction image is obtained at every 10 minutes from a reaction start time (0 minute) to the result time (15 minutes).

At this time, some of all reaction images (reaction image 1 to reaction image n in FIG. 10) over time and all actual concentrations (actual concentration 1 to actual concentration n in FIG. 10) over time are used as learning data and the remaining is used as test data and validation data. For example, odd-numbered reaction images and actual concentrations are used as training data to train the analysis result prediction model. Even-numbered reaction images and actual concentrations are used as test data and validation data to test and validate the analysis result prediction model.

Further, a pre-processing process of the reaction image is performed before inputting the reaction image to the analysis result prediction model. For example, as illustrated in FIG. 12, a reaction image of an area corresponding to the test-line is obtained from the entire images.

Now, the implementation example of the deep learning-based analysis result predicting device 100 according to a preferred embodiment of the present invention will be described.

First, a reaction image of an initial period (for example, “0 minute to 5 minutes” for interaction of a sample obtained from the specimen and an optical-based kit (for example, a lateral flow assay kit) is obtained from an imaging device (not illustrated). At this time, the camera photographs in a predetermined time unit (for example, “10 seconds”) to obtain a plurality of reaction images. The camera may capture a video during an initial period and extract an image frame from the captured video in a predetermined time unit to obtain a plurality of reaction images.

Thereafter, the camera provides the reaction image to the analysis result predicting device 100 according to the present invention directly or via an external server, through wireless/wired communication. When the analysis result predicting device 100 according to the present invention includes a capturing module, the analysis result predicting device may directly obtain the reaction image.

The analysis result predicting device 100 according to the present invention predicts a concentration for the result time (for example, “15 minutes” using the previously stored analysis result prediction model on the basis of the reaction image of the initial period and outputs the result. The analysis result predicting device 100 according to the present invention also provides the reaction image of the initial period to an external server in which the analysis result prediction model is stored through the wireless/wired communication and receives the predicted concentration for the result time from the external server to output the result.

According to another embodiment of the present invention, the analysis result prediction technique of the present invention may be used for an on-site diagnostic kit. Specifically, the techniques described in the specification of the present invention may be implemented as a prediction diagnostic device for an on-site diagnostic test.

The present invention relates to a prediction diagnostic device for an on-site diagnostic test. Here, the prediction diagnostic device includes: a memory in which instructions required for on-site diagnosis are stored, and a processor which performs operations for prediction diagnosis according to the execution of the instructions and the operations includes: a step of applying a sample obtained from a specimen to a diagnostic kit and obtaining an initial reaction image of a predetermined initial period according to the interaction of the sample and the diagnostic kit; and a step of predicting a result reaction of a result period after the initial period by applying the reaction image to a pre-trained and established analysis result prediction model.

Further, the analysis result prediction model of the present invention includes an artificial neural network which applies a training sample obtained from a training specimen to the diagnostic kit and is trained using a plurality of time-series reaction images according to the interaction of the training sample obtained over time and the diagnostic kit.

In the present invention, the plurality of time-series reaction images includes: a first reaction image at a first timing belonging to the predetermined initial period and a second reaction image at a second timing which belongs to the predetermined initial period and follows the first timing.

The analysis result prediction model of the present invention further includes an artificial neural network configured to adaptively update a current state value according to the second reaction image, using a previous state value corresponding to the first reaction image.

Further, the analysis result prediction model includes an encoder and the encoder includes a long short-term memory (LSTM) type artificial neural network, and a convolutional neural network (CNN) which extracts a feature value from a reaction image at the first timing and a reaction image at the second timing. The feature value extracted from the convolution neural network may be used as an input of the long short-term memory type artificial neural network.

The analysis result prediction model of the present invention further includes a first regression model which generates a feature value corresponding to a result reaction of a result period as the result reaction using a latent vector obtained from the LSTM and the operations performed by the processor further include a step of predicting a concentration of a target material included in the sample using the generated feature value.

Further, the analysis result prediction model further includes a decoder and the decoder includes a generative adversarial network (GAN) and a second regression model. The generative adversarial network generates a result image at a timing corresponding to the result period using the latent vector obtained from the LSTM and the second regression model generates a feature corresponding to the result reaction of the result period as the result reaction using the generated result image.

The operations performed by the processor further include a step of predicting a concentration of a target material included in the sample using the feature value generated in the second regression model. Further, the second regression model is trained using the learning data to minimize the difference of the predicted concentration for the predetermined result time obtained on the basis of the reaction image of the learning data and the actual concentration for the predetermined result time of the learning data.

The initial reaction image is an image according to an interaction of the sample and the diagnostic kit and includes a test-line and a control-line.

The processor pre-processes the initial image to minimize the influence according to external factors included in the initial reaction image and then applies the pre-processed image to the analysis prediction model. Here, the pre-processing of the initial image includes cropping of an image in an area of the test-line and the control-line or reducing an effect according to external illumination or reducing a spatial bias of the initial image, or adjusting a scale of the initial image.

Prominent techniques such as real-time polymerase chain reaction (RT-PCR), enzyme-linked immunosorbent assay (ELISA), and rapid kits are currently being explored to enhance sensitivity and reduce assay time. Existing commercial molecular diagnostic methods typically take several hours, while immunoassays can range from several hours to tens of minutes. Rapid diagnostics are crucial in Point-of-Care Testing (POCT). We propose an approach that integrates a time-series deep learning architecture, AI-based verification, and enhanced result analysis. This approach is applicable to both infectious diseases and non-infectious biomarkers. In blind tests using clinical samples, our method achieved diagnostic times as short as 2 minutes, exceeding the accuracy of human analysis at 15 minutes. Furthermore, our technique significantly reduces assay time to just 1 minute in the POCT setting. This advancement has considerable potential to greatly enhance POCT diagnostics, enabling both healthcare professionals and non-experts to make rapid, accurate decisions.

In Point-of-Care Testing (POCT), achieving both high sensitivity and affordable rapid diagnosis is a pivotal challenge. POCT methods are broadly categorized into immunoassay-based and molecular-based approaches. Recent advancements in molecular diagnostics have shown the potential to reduce assay time to less than 10 minutes using plasmonics and microfluidic techniques. However, in the case of most commercialized molecular diagnostics, a sample preparation step is inevitably involved, leading to a relatively lengthy diagnosis time of up to several hours.

On the other hand, in immunoassay-based diagnostics, short detection times based on nanosensors, such as nanowires and field-effect transistor (FET) sensors, have been reported; however, few have received FDA approval. The commercialized immunoassay platform encompasses enzyme-linked immunosorbent assay (ELISA), fluorescence Immunoassay (FIA), chemiluminescent immunoassay (CLIA), and lateral flow assay (LFA). ELISA, as the most popular immunoassay platform, requires a significant amount of time, approximately 3 to 5 hours for analysis. In contrast, rapid kits, also known as rapid diagnostic tests (RDT), provide quicker results, typically within 15 minutes, providing the fastest immunoassay.

In the domain of emergency medical care, expeditious and precise diagnosis within the emergency room (ER) holds utmost significance. The patients arriving at the ER often present with severe, life-threatening, or time-sensitive conditions, necessitating prompt and accurate diagnostic interventions. For example, cardiac troponin I, which is highly specific to myocardial tissue and undetectable in healthy individuals, is significantly elevated in patients with myocardial infarction and can remain elevated for up to 10 days post-necrosis. Levels above 0.4 ng/ml indicate a notably higher 42-day mortality risk. Particularly for myocardial infarction patients who present to the emergency room, prompt diagnosis and management are crucial. In such critical scenarios, the rapid identification of diseases and conditions exerts a profound impact on patient outcomes.

Notably, in cases involving infectious diseases, timely diagnosis plays a pivotal role in identifying the causative pathogens and infections, thereby facilitating the timely implementation of infection control measures to avert potential outbreaks and safeguard the health of both patients and healthcare providers.

Furthermore, for pregnant patients in the ER, knowing their pregnancy status is crucial, especially when considering medical imaging involving radiation, anesthesia, or treatments that could affect fetal well-being. Fast and precise diagnosis is key in guiding informed decisions, enabling the effective management of health conditions while simultaneously minimizing risks to both the patient and the fetus.

While LFA is generally recognized as a rapid and commercially viable diagnostic tool, its significance in enabling timely interventions extends beyond its immediate applications.

LFA also holds a pivotal role in reducing unnecessary tests and treatments, thereby contributing to more efficient healthcare utilization and cost-effectiveness. Consequently, the approaches to further shorten assay time while retaining sensitivity have elicited considerable interest, given its potential to unlock numerous novel detection opportunities. These advancements show promise, particularly in emergency medicine, infectious disease management, and neonatal care, with the potential to improve patient outcomes.

Artificial intelligent (AI) technology has emerged as a focal point in medical image-based diagnostics using convolution neural networks (CNN), encompassing modalities such

X-ray, computed tomography (CT), and magnetic resonance imaging (MRI), with its application promising significant enhancements in diagnostic accuracy while revolutionizing the interpretation and analysis of complex medical images. Recently, our group proposed deep learning-assisted smart phone-based LFA (SMARTAI-LFA) and demonstrated that integrating clinical sample learning and two-step algorithms enables a cradle-free on-site assay with higher accuracy (>98%). However, the earlier study primarily highlighted the performance of AI-enhanced colorimetric assays and did not specifically address the reduction of assay time using AI.

Several recent studies in medical diagnostics have emphasized the reduction times by integrating deep learning techniques. Innovative studies have successfully achieved shorter histopathology tissue staining times using generative adversarial network (GAN)-based virtual staining and applied deep learning methodologies to enhance efficiency in plaque assays.

Moreover, the utilization of long short term memory (LSTM) deep learning algorithms has expedited polymerase chain reaction (PCR) analysis, enabled the prediction of infections based on time-series data from affected individuals, and facilitated the utilization of longitudinal MRI images for predicting treatment responses. Meanwhile, the demand for diagnostic tools achieving shortened assay time and maintained sensitivity remains high, but few studies address for achieving AI-assisted fast assay, especially for POCT. Consequently, there is a pressing need for AI technology to enable rapid diagnosis in POCT, representing a transformative step in enhancing diagnostic efficiency beyond traditional hardware optimization.

In the present invention, we present an innovative approach that combines a time-series deep learning algorithm with lateral flow assay platforms, notably the most affordable and accessible POCT platform, to achieve a significant reduction in assay time, now as short as 1 minute. Our method, which utilizes an architecture comprising YOLO, CNN-LSTM, and a fully connected (FC) layer, notably accelerates the COVID-19 Ag rapid kit's assay time, facilitated by the Time-Efficient Immunoassay with Smart AI-based Verification (TIMESAVER). This approach is versatile, applicable to a range of conditions including infectious diseases like COVID-19 and Influenza, as well as non-infectious biomarkers such as Troponin I and hCG. In blind tests with clinical samples, our method not only achieved diagnostic times as short as 2 minutes but also surpassed the accuracy of human analysis traditionally completed in 15 minutes.

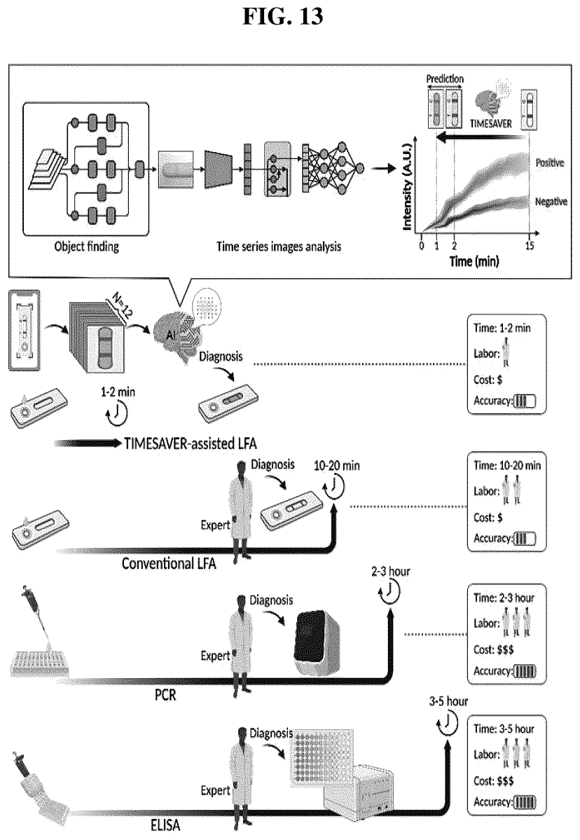

FIG. 13 presents three representative commercialized diagnostic tools: commercial LFA, PCR, and ELISA, along with their performance in terms of time, labor, cost, and accuracy. Generally, commercial PCR and ELISA tests take several hours, are labor-intensive, and incur higher costs. In contrast, rapid kits typically provide cost-effective, on-site diagnostics. We introduce TIMESAVER-assisted LFA, a comprehensive approach that combines time-series deep learning architecture, AI-based verification, and enhanced result analysis to optimize LFA immunoassays. Our objective is to establish the fastest diagnostic time among existing commercially available kits while maintaining accuracy and affordability. Conventional rapid kit protocols typically require 10 to 20 minutes for analysis, posing challenges in time-sensitive applications like emergency medicine, infectious disease management, neonatal care, and heart stroke, where further assay time reduction is crucial.

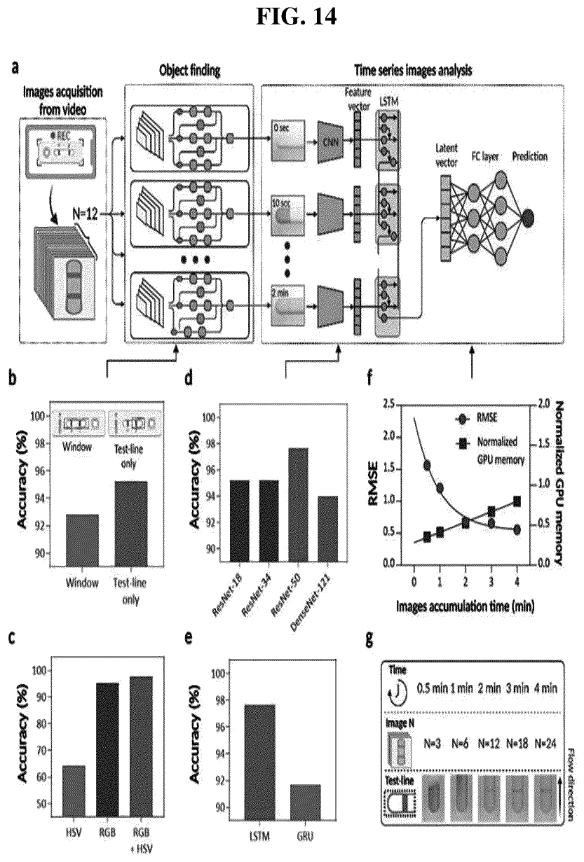

As shown in FIG. 13, our approach utilizes a time-series deep learning architecture and AI-based verification, resulting in a significant reduction in assay time to within 1-2 minutes using TIMESAVER. A more detailed discussion of the time-series deep learning architecture, known as the TIMESAVER algorithm, is provided in FIG. 14. This algorithm is specifically designed for learning from time-series data and has effectively reduced diagnosis times. Notably, the results demonstrate diagnosis times as short as 1-2 minutes for LFA when utilizing a smartphone or reader.

### Model Optimization for TIMESAVER Algorithm

FIG. 14, illustrates an example of a deep learning architecture, TIMESAVER, utilized for predicting results, which consists of three components: YOLO, CNN-LSTM, and the FC layer. FIG. 14a illustrates the overall scheme of TIMESAVER, a deep learning architecture consisting of three interconnected components. This involves transforming the entire image into a cropped image containing the test line, which is then processed through CNN and LSTM networks to generate a vector representation. Subsequently, the CNN and LSTM outputs are combined and passed through the FC layer to produce the predicted result.

Region of Interest (ROI) selection is a crucial step in rapid kit diagnosis (FIG. 14b). The selection of the Region of Interest (ROI) enhances the accuracy of detecting the specific concentration of the target biomarker or pathogen, thereby increasing sensitivity and specificity and minimizing the occurrence of false negatives and false positives. As detailed in our previous research, we investigated two methods for ROI selection in LFAs: focusing on the window and the test line exclusively. The approach centered on the window area achieved a prediction accuracy of 92.9%, while a focus exclusively on the test line enhanced the prediction accuracy to 95.2%. Data augmentation is a vital technique, particularly for limited or imbalanced datasets (FIG. 14c). It involves applying various transformations to existing data, generating synthetic images to enrich the dataset and enhance the model's robustness. In our study, we acquired RGB channel images and transformed them into HSV channel images. The data augmentation results were as follows: RGB achieved an accuracy of 95.2%, HSV achieved 64.3%, and combining RGB and HSV yielded a perfect accuracy of 97.6%.

FIG. 14d illustrates an optimized CNN model. For feature extraction from images, we used a CNN specifically designed for image recognition and processing tasks, making CNNs essential in computer vision applications. Among the four frameworks evaluated (ResNet-18, ResNet-34, ResNet-50, DenseNet-121), ResNet-50 exhibited the highest accuracy at 97.6%, surpassing the performance of shallow-layer models. FIG. 14e illustrates an optimized LSTM model. When forecasting using time-series data, we employed advanced recurrent neural network (RNN) algorithms, including LSTM and gated recurrent unit (GRU). LSTM, a type of recurrent neural network, excels in handling sequential data and addresses the vanishing gradient problem by employing a sophisticated memory cell. LSTM achieved an accuracy of 97.6%, while GRU obtained 91.7%.

FIG. 14f shows the trade-off curve between root mean squared error (RMSE) and normalized graphics processing unit (GPU) memory consumption across various assay time frames, effectively illustrating the AI-based optimized assay time. Note that assay time refers to the sequential images used in training and testing. As we incorporated additional time-series data, the RMSE values were exponentially reduced, indicating enhanced accuracy. However, this improvement was accompanied by a linear increase in GPU memory consumption. Controlling GPU memory consumption is a key parameter for achieving optimal deep learning operation, as higher GPU memory consumption leads to longer training/test times and requires more expensive hardware. Consequently, we postulate that a 2-minute time series may represent the optimal condition when employing the TIMESAVER model.

FIG. 14g shows the acquired images over time. After approximately 30 seconds, the samples loaded in the sample reservoir reached the test line, and the test line appeared after 1 to 2 minutes, depending on the concentration/titers of the target. All the images were taken at 10-second intervals, resulting in 6 images acquired per minute. For example, in a 2-minute assay, we trained on 12 sequential images, then tested sequential images with a 2-minute assay time. Interestingly, in the time scale of 1 to 2 minutes, we observed unclear background signals with the naked eye; however, the TIMESAVER model could detect the colorimetric signal with higher accuracy.

### Assay of Infectious Diseases Via TIMESAVER

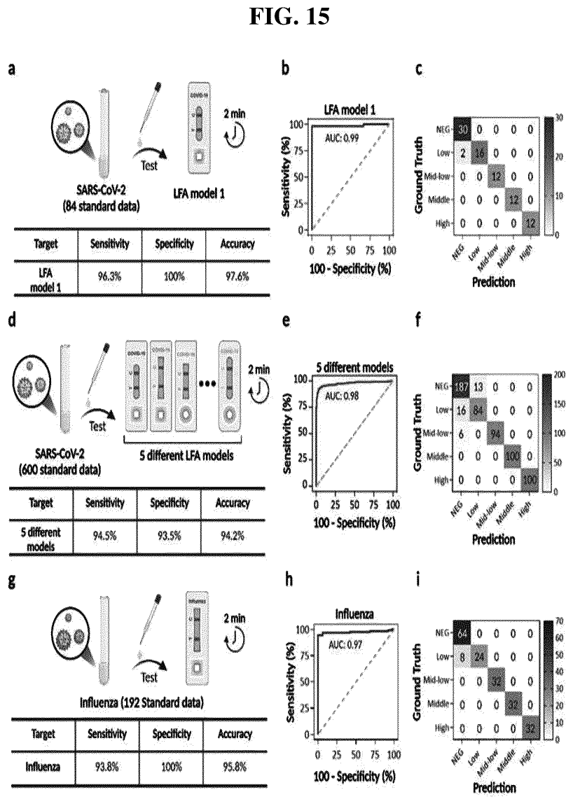

FIG. 15 presents the assessment of infectious diseases, specifically COVID-19 antigen and Influenza A/B, using a 2-minute assay facilitated by the TIMESAVER model. To assess the diagnostic accuracy of COVID-19 in FIG. 15a, we employed standard data (target protein spiked rapid kit running buffer) and trained the TIMESAVER model using our training set, which included both the training data (n=594) and a validation subset (10% of the training set). We developed a regression model for TIMESAVER and categorized the regression values into five classes: high, middle, mid-low, low, and negative control. It's important to note that we categorized the images from data into these classes following the manufacturer's supplied guidelines, which are as follows: high (levels 8-7), middle (levels 6-5), mid-low (levels 4-3), low (levels 2-1), and negative control (level 0). Consequently, we can conduct a more comprehensive examination of the underlying causes of false positive and false negative signals. Since each dataset comprises 12 time frame images with 10-second intervals, the total number of images used for training was 7,128. We conducted tests with 84 data (54 positive and 30 negative). Our results indicate that the AI-based decision-making process, performed within 2 minutes, achieved a sensitivity of 96.3%, specificity of 100%, and accuracy of 97.6%, showcasing the excellence of the TIMESAVER model in making initial decisions.

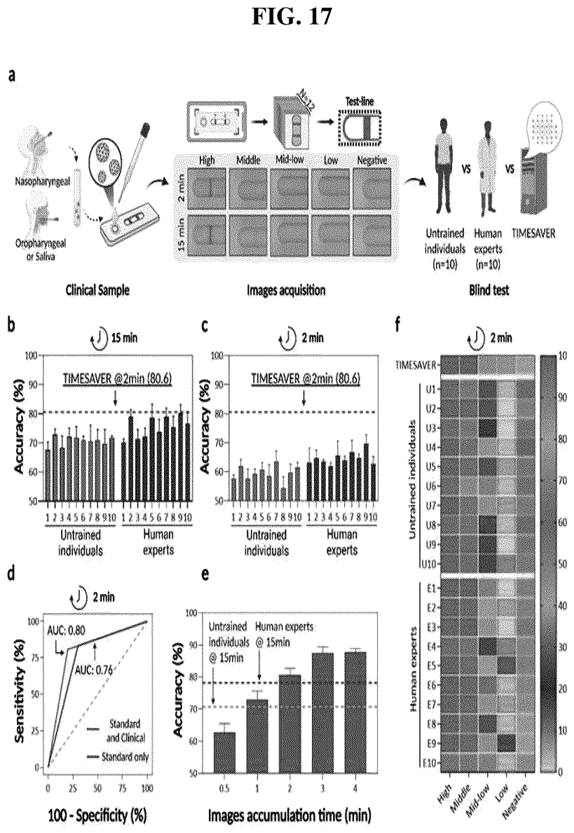

FIG. 15b-c show receiver operating characteristic (ROC) curves and a confusion matrix for the 2-minute assay of COVID-19 using the TIMESAVER algorithm. ROC curves provide a comprehensive view of the model's performance, with a higher area under the curve (AUC) indicating better classification ability. Our analysis revealed that the TIMESAVER model achieved an impressive AUC of 0.99, affirming its excellence as an assay classifier. The confusion matrix (FIG. 15c) highlights the critical nature of accurately diagnosing low-concentration data, a challenge even for experts when relying solely on visual inspection. We observed that the false negatives (n=2) were caused by the low-concentration samples. This provides valuable insights for improving sensitivity and specificity. One viable strategy involves augmenting the training data. By incorporating more data with low concentrations, we can fine-tune sensitivity and specificity, as demonstrated in our previous paper39. In the following section, we will demonstrate the enhanced accuracy of our clinical assay. This will be achieved by integrating clinical data from 84 patients, including 13 with Ct values>29, corresponding to low concentration/titer, and 32 healthy controls, as detailed in FIG. 17.

Universality is a key characteristic of the TIMESAVER algorithm. We validated its universality by assessing its performance on various commercialized LFA models (FIG. 15d-f). In this study, we tested an additional five LFA models (n=600, FIG. 15d, We exclusively trained the TIMESAVER model with an additional set of time-series data (n=300) combined with the pre-existing dataset (n=594), resulting in a total training dataset of 894. Given that each dataset consists of 12 time frame images, the total number of images used for training amounted to 10,728. To test the algorithm, we applied the TIMESAVER model initially trained with LFA model 1 (COVID-19 Ag LFA kits, Calth Inc.). Interestingly, the average sensitivity and specificity across these five different models (n=600), each with distinct form factors, were 94.5% and 93.5%, respectively. The variation in performance can be attributed to differences in membrane types, designs, materials, flow rates, and other factors among LFA kits from various manufacturers. Such variations are expected due to the hardware-related disparities between these different LFAs. The AUC value reached 0.98 as shown in the ROC curve (FIG. 15e). Furthermore, from the confusion matrix (FIG. 15f), it is evident that the ability to discriminate lower concentrations and negative controls plays a pivotal role in LFA assays for achieving higher accuracy. We anticipate that further training with various LFA models will lead to increased accuracy, as demonstrated in our previous works.

We broadened our validation efforts to include influenza testing. The influenza kit in our study had A, B, and control lines, but due to limited sample availability, we only tested for influenza A. Illustrated in FIG. 15g-h, the manuscript details the sensitivity, specificity, and accuracy in detecting Influenza A, based on a dataset of 192 test samples. The influenza test kits exhibited a sensitivity of 93.8%, specificity of 100%, and an accuracy of 95.8%. The AUC value derived from the ROC curve was 0.97. it was observed that the false negatives (n=8) were predominantly due to samples with low concentrations, which adversely affected sensitivity. However, the Lateral Flow Assay (LFA) enhanced by the TIMESAVER model demonstrated that it is possible to achieve a quick assay time while still maintaining the essential sensitivity and specificity for effective point-of-care diagnosis.

### Assay of Non-Infectious Biomarkers for Emergency Room (ER) Via TIMESAVER

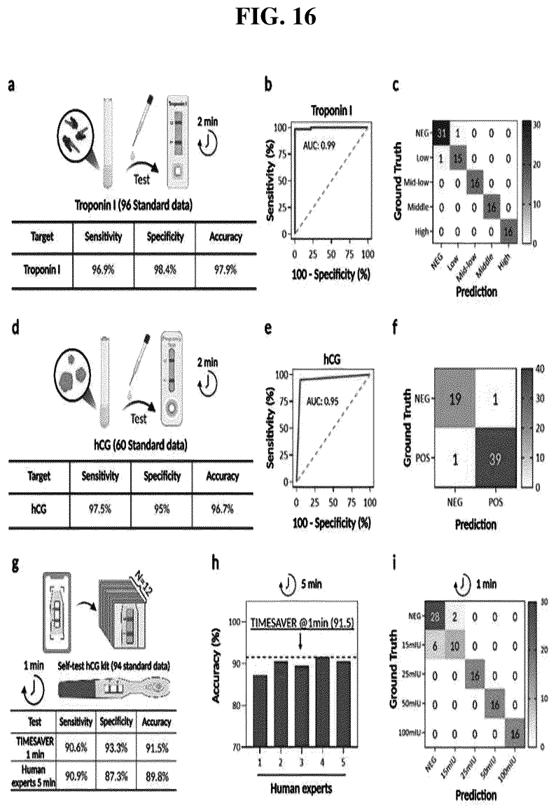

Next, we further validated the performance of the TIMESAVER assay for non-infectious biomarkers, including Troponin I and hCG for ER. Initially focusing on Troponin I, as shown in FIG. 16a-c, we acknowledged its clinical relevance above 0.4 ng/ml, following previous research. Therefore, we set a cut-off at 0.5 ng/mL and established a five-class multi-classification using recombinant protein, based on LFA manufacturer's guideline. This involved training with 618 data, validation with 62 data, and testing with 96 data. The results yielded a sensitivity of 96.9%, specificity of 98.4%, and accuracy of 97.9% (FIG. 16a). In FIG. 16b, the AUC value from the ROC curve was 0.99, and the TIMESAVER demonstrated high accuracy within a 2-minute diagnostic timeframe. TIMESAVER showed some false signals at lower concentrations (FIG. 16c), which appear to be more a limitation of the LFA rather than the algorithm. These results confirm the effectiveness of our algorithm in achieving multi-classification within just 2 minutes of testing, underscoring its utility in rapid diagnostic scenarios.

In emergency room settings, rapid diagnosis of hCG is essential, particularly for assessing pregnancy in patients. (FIG. 16d) demonstrates the sensitivity, specificity, and accuracy for hCG detection within 2 minutes, using test data (n=60). The results revealed that the sensitivity, specificity, and accuracy for hCG were 97.5%, 95.0%, and 96.7%, respectively. The AUC value derived from the ROC curve was 0.95 (FIG. 16e), and the confusion matrix (FIG. 16f) suggests effective performance of the classifier, even when applied in a 2-minute assay utilizing the TIMESAVER model.

We aimed to assess the feasibility of achieving the fastest assay within 1 minute among commercially available diagnostic tests (FIG. 16g-i). Generally, hCG self-tests exhibit rapid flow velocity, and signal readings are typically recommended after a 5-minute wait according to the manufacturer's guidelines. In our primary training data (n=594), initially trained for COVID-19, we incorporated an additional hCG dataset (n=24), resulting in a total training set of 618 data (FIG. 16g). The hCG dataset consisted of 30 images captured at 2-second intervals. We then used 12 images taken between 36 to 60 seconds. The test dataset consisted of 94 standard data. Even with a 1-minute assay facilitated by TIMESAVER, we achieved impressive results with a sensitivity of 90.6%, specificity of 93.3%, and an overall accuracy of 91.5%. The sensitivity, specificity, and overall accuracy of five human experts at 5 minutes were 90.9%, 87.3%, and 89.8%, respectively. In FIG. 16h, we observed that the accuracy with TIMESAVER at 1 minute surpassed the accuracy of five experts at 5 minutes. As anticipated, false positives and false negatives of TIMESAVER at 1 minute were primarily associated with lower concentrations (15 mIU), particularly those near the cutoff threshold (FIG. 16i).

### Blind Tests Using Clinical Samples

FIG. 17 illustrates the clinical evaluation of COVID-19 through blind tests. We assessed the blind tests from three different groups: untrained individuals, human experts, and TIMESAVER, utilizing 252 test data (156 positives and 96 negatives). Clinical samples were collected from COVID-19 patients at Seoul St. Mary's Hospital, including information on SARS-CoV-2 patients (n=52) and healthy controls (n=32). The 252 test data come from the three different rapid kit tests performed on COVID-19 patients (n=84). This information encompassed sample collection details, variants, sex, ages, and Ct values. All samples underwent RT-qPCR analysis, followed by the LFA assay. The data from the LFA assay were classified into five groups: high/middle/middle-low/low titer, and negative control, using a color chart level (high with levels 8-7, middle with levels 6-5, middle-low with levels 4-3, and low titer with levels 2-1 for positive, and negative with level 0). Among the positive data (n=156), we distributed the data across four groups (high: 30, middle: 48, mid-low: 39, low: 39). We also included negative data from healthy controls (n=96).

For the blind test, ten untrained individuals and ten human experts each tested 252 data, including 30 high, 48 middle, 39 middle-low, 39 low, and 96 negative data. This resulted in a total of 5040 blind tests for both untrained individuals and human experts. As shown in FIG. 17a, the colorimetric assay results were captured using a custom-made charge-coupled device (CCD) camera, or potentially a smartphone camera, displaying clear positive images in high and middle concentrations. However, below the mid-low concentration, no distinct positive signal could be captured. Interestingly, the assay conducted within 2 minutes exhibited a larger background signal, which hindered the clear observation of the colorimetric signal by the naked eye.

We presented the results of blind tests using images from a 15-minute assay (FIG. 17b) followed by a 2-minute assay (FIG. 17c), involving both untrained individuals and human experts, as well as the TIMESAVER algorithm, which demonstrated a notable reduction in assay time. The 15-minute assay shown in FIG. 5b was conducted following the manufacturer's guidelines for conventional assays. In these 15-minute assay images, untrained individuals reached an accuracy rate of 70.7%, while human experts attained 78.1%. The lower accuracy compared to the manufacturer's claim of >90% sensitivity and >99% specificity can be attributed to our inclusion of a substantial number of lower titer data. Nevertheless, the TIMESAVER model surpassed both human experts and untrained individuals in performance, achieving a higher accuracy of 80.6% even in a shortened 2-minute assay.

When the assay time was reduced to 2 minutes (FIG. 17c), identifying clear positive signals for mid-low concentrations became problematic for the naked eye, and the reddish background often led to more false positives. As a result, the accuracy rates for untrained individuals and human experts fell to 59.4% and 64.6%, respectively. In contrast, the TIMESAVER algorithm maintained a high accuracy of 80.6% in the 2-minute assay. While the accuracy of human interpretation significantly decreased at lower concentrations (lower viral load), indicating a tendency for human error in rapid assessments, the AI-driven TIMESAVER algorithm showed greater precision, effectively handling background noise and unclear colorimetric signals. This allowed for fast assays with improved accuracy, showcasing the potential of AI in enhancing rapid diagnostic techniques.

FIG. 17d displays the influence of clinical training data on ROC curves. Initially, we present ROC curves trained with a standard dataset (n=594, shown in blue and labeled as ‘standard only’). We then demonstrate improved ROC curves achieved after additional training with clinical data (n=694, shown in red and labeled as ‘standard and clinical’). The ROC curve is a widely-used tool for assessing the clinical effectiveness of diagnostic models. The AUC with the inclusion of clinical data (0.80) exceeded that with the standard dataset alone (0.76). Although the TIMESAVER algorithm with a 2-minute assay might not entirely match the accuracy standards of clinical laboratories, its ability to continuously improve diagnostic accuracy through learning from acquired images is notable. By further incorporating deep learning with clinical samples, we can enhance the clinical accuracy of our diagnostic approach.

We demonstrate the capability of TIMESAVER to achieve accuracy levels comparable to those of human experts in the shortest possible time frame (FIG. 17e). We initiated the assay timer when the sample was introduced into the sample reservoir, capturing sequential images over time. We established five distinct datasets, each representing varying assay durations (0.5, 1, 2, 3, and 4 minutes). For example, in the case of a 1-minute assay, we obtained 6 images with 10-second intervals. Our TIMESAVER model demonstrated that it requires only 1 minute to attain accuracy equivalent to that achieved by untrained individuals. With a 2-minute assay, we achieved an accuracy rate of 80.6%, surpassing the accuracy of human experts at the 15-minute mark (78.1%). The samples reached the test line within 1 minute, enabling the AI to precisely ascertain the assay results during the initial color development phase. In comparison to conventional human-conducted assays, where TIMESAVER completes the assay in just 2 minutes, it consistently outperforms human experts in terms of accuracy.

The heat map indicates that human visual assessment, conducted by both untrained individuals and experts, shows a decrease in accuracy, particularly within the mid-low titer ranges (FIG. 17f). In the mid-low titer category, untrained individuals managed an average accuracy of only 29.2%, while human experts fared slightly better at 37.2%. In contrast, our algorithm achieved an impressive accuracy rate of 84.6%. For the low titer category, the accuracy was even lower, with untrained individuals at 2.8% and human experts at 5.4%, but our deep learning algorithm significantly outperformed at 38.5% accuracy. In cases of high and middle titer concentrations, the TIMESAVER algorithm consistently provided reliable and accurate data, effectively eliminating the variability seen in human visual assessments.

The operation according to the embodiment of the present disclosure may be implemented as a program instruction which may be executed by various computers to be recorded in a computer readable storage medium. The computer readable storage medium indicates an arbitrary medium which participates to provide a command to a processor for execution. The computer readable storage medium may include solely a program command, a data file, and a data structure or a combination thereof. For example, the computer readable medium may include a magnetic medium, an optical recording medium, and a memory. The computer program may be distributed on a networked computer system so that the computer readable code may be stored and executed in a distributed manner. Functional programs, codes, and code segments for implementing the present embodiment may be easily inferred by programmers in the art to which this embodiment belongs.

The present embodiments are provided to explain the technical spirit of the present embodiment and the scope of the technical spirit of the present embodiment is not limited by these embodiments. The protection scope of the present embodiments should be interpreted based on the following appended claims and it should be appreciated that all technical spirits included within a range equivalent thereto are included in the protection scope of the present embodiments.

### EXPLANATION OF REFERENCE NUMERALS AND SYMBOLS

100: Analysis result predicting device
110: Processor
130: Computer readable storage medium
131: Program
150: Communication bus
170: Input/output interface
190: Communication interface

1. A deep learning-based analysis result predicting method, comprising:
a step of obtaining a reaction image for a predetermined initial period for an interaction of a sample obtained from a specimen and an optical-based kit; and
a step of predicting a concentration for a predetermined result time on the basis of the reaction image for the predetermined initial period, using a pre-trained and established analysis result prediction model.

2. The deep learning-based analysis result predicting method of claim 1, wherein the step of obtaining a reaction image is configured by obtaining a plurality of reaction images in a predetermined time unit for the predetermined initial period.

3. The deep learning-based analysis result predicting method of claim 2, wherein the analysis result prediction model includes:
an image generator includes a convolution neural network (CNN), long short-term memory (LSTM), and a generative adversarial network (GAN), and generates a prediction image corresponding to a predetermined result time on the basis of an input reaction image, and outputs the generated prediction image; and
a regression model which includes the convolution neural network (CNN) and outputs a predicted concentration for a predetermined result time on the basis of the prediction image generated by the image generator, and
the regression model is trained using the learning data to minimize the difference of the predicted concentration for the predetermined result time obtained on the basis of the reaction image of the learning data and the actual concentration for the predetermined result time of the learning data.

4. The deep learning-based analysis result predicting method of claim 3, wherein the image generator includes:
an encoder which obtains a feature vector from the input reaction image using the convolution neural network (CNN), obtains a latent vector on the basis of the obtained feature vector using the long short term memory (LSTM), and outputs the obtained latent vector; and
a decoder which generates the prediction image on the basis of the latent vector obtained from the encoder using the generative adversarial network (GAN), and outputs the generated prediction image.

5. The deep learning-based analysis result predicting method of claim 4, wherein the decoder includes:
a generator which generates the prediction image on the basis of the latent vector and outputs the generated prediction image; and
a discriminator which compares the prediction image generated by the generator and an actual image corresponding to a predetermined result time of the learning data and outputs a comparison result, and
it is trained to discriminate that the prediction image obtained on the basis of the latent vector is the actual image using the learning data.

6. The deep learning-based analysis result predicting method of claim 1, wherein the step of obtaining a reaction image is configured by obtaining the reaction image of an area corresponding to the test-line when the optical-based kit includes a test-line and a control-line.

7. The deep learning-based analysis result predicting method of claim 6, wherein the step of obtaining a reaction image is configured by obtaining the reaction image of the area corresponding to one or more predetermined test-lines, among a plurality of test-lines when the optical-based kit includes a plurality of test-lines.

8. The deep learning-based analysis result predicting method of claim 7, wherein the step of obtaining a reaction image is configured by obtaining the reaction image including all areas corresponding to one or more predetermined test-lines, among the plurality of test-lines or obtaining the reaction image for every test-line to distinguish areas corresponding to one or more predetermined test-lines, among the plurality of test-lines for every test-line.

9. A deep learning-based analysis result predicting device which predicts an analysis result based on deep learning, comprising:
a memory which stores one or more programs to predict an analysis result; and
one or more processors which perform an operation for predicting the analysis result according to one or more programs stored in the memory,
wherein the processor predicts a concentration for a predetermined result time on the basis of a reaction image of a predetermined initial period for an interaction of a sample obtained from a specimen and an optical-based kit, using a pre-trained and established analysis result prediction model.

10. The deep learning-based analysis result predicting device of claim 9, wherein the processor obtains a plurality of reaction images in a predetermined time unit for the predetermined initial period.

11. The deep learning-based analysis result predicting device of claim 10, wherein the analysis result prediction model includes:
an image generator includes a convolution neural network (CNN), long short-term memory (LSTM), and a generative adversarial network (GAN), and generates a prediction image corresponding to a predetermined result time on the basis of an input reaction image, and outputs the generated prediction image; and
a regression model which includes the convolution neural network (CNN) and outputs a predicted concentration for a predetermined result time on the basis of the prediction image generated by the image generator, and
the regression model is trained using the learning data to minimize the difference of the predicted concentration for the predetermined result time obtained on the basis of the reaction image of the learning data and the actual concentration for the predetermined result time of the learning data.

12. The deep learning-based analysis result predicting device of claim 11, wherein the image generator includes:
an encoder which obtains a feature vector from the input reaction image using the convolution neural network (CNN), obtains a latent vector on the basis of the obtained feature vector using the long short term memory (LSTM), and outputs the obtained latent vector; and
a decoder which generates the prediction image on the basis of the latent vector obtained from the encoder using the generative adversarial network (GAN), and outputs the generated prediction image.

13. A prediction diagnostic device for an on-site diagnostic test, comprising:
a memory in which instructions required for on-site diagnosis are stored; and
a processor which performs operations for prediction diagnosis according to the execution of the instructions and the operations includes:
a step of applying a sample obtained from a specimen to a diagnostic kit and obtaining an initial reaction image of a predetermined initial period according to the interaction of the sample and the diagnostic kit; and
a step of predicting a result reaction of a result period after the initial period by applying the reaction image to a pre-trained and established analysis result prediction model.

14. The prediction diagnostic device for an on-site diagnostic test of claim 13, wherein the analysis result prediction model includes an artificial neural network which applies a training sample obtained from a training specimen to the diagnostic kit and is trained using a plurality of time-series reaction images according to the interaction of the training sample obtained over time and the diagnostic kit.

15. The prediction diagnostic device for an on-site diagnostic test of claim 14, wherein the plurality of time-series reaction images includes:
a first reaction image at a first timing belonging to the predetermined initial period and
a second reaction image at a second timing which belongs to the predetermined initial period and follows the first timing.

16. The prediction diagnostic device for an on-site diagnostic test of claim 14, wherein analysis result prediction model includes an encoder and the encoder includes:
a long short-term memory (LSTM) type artificial neural network, and
a convolutional neural network (CNN) which extracts a feature value from a reaction image at the first timing and a reaction image at the second timing,
the feature value extracted from the convolution neural network is used as an input of the long short-term memory type artificial neural network.
